(19) United States
(12) Patent Application Publication
(10) Pub. No.: US 2020/0367800 A1
(43) Pub. Date: Nov. 26, 2020

(54) METHOD FOR IDENTIFYING DRIVING FATIGUE BASED ON CNN-LSTM DEEP LEARNING MODEL

(71) Applicant: WUYI University, Guangdong (CN)

(72) Inventors: Hongtao WANG, Guangdong (CN); Xucheng LIU, Guangdong (CN); Cong WU, Guangdong (CN); Cong TANG, Guangdong (CN); Zi An PEI, Guangdong (CN); Hongwei YUE, Guangdong (CN); Peng CHEN, Guangdong (CN); Ting LI, Guangdong (CN)

(21) Appl. No.: 16/629,931

(22) PCT Filed: Mar. 22, 2019
(86) PCT No.: PCT/CN2019/079258

§ 371 Date: Jul. 30, 2020

(30) Foreign Application Priority Data
Jan. 23, 2019 (CN) 201910063299.8

(51) Int. Cl. A61B 5/18; A61B 5/04; A61B 5/00; A61B 5/0484; A61B 5/16; A61B 5/0402

(52) U.S. Cl. CPC A61B 5/18; A61B 5/04004; A61B 5/7264; G06N 3/04; A61B 5/7225; A61B 5/162; A61B 5/04021; A61B 5/0484

(57) ABSTRACT

Disclosed is a method for identifying driving fatigue based on a CNN-LSTM deep learning model including: collecting electroencephalograph signals of a subject during simulated driving; randomly issuing an operating command during simulated driving, and dividing the electroencephalograph signals into fatigue data and non-fatigue data according to a reaction time for the subject to complete the operating command; performing band-pass filtering and mean removal preprocessing on the electroencephalograph signals, and respectively extracting N minutes of fatigue electroencephalograph signal data and N minutes of non-fatigue electroencephalograph signal data to be detected; performing independent component analysis on the electroencephalograph signal data to remove interference signals; establishing a CNN-LSTM model and setting network parameters of the CNN-LSTM model; transmitting the electroencephalograph signal data with interference signals removed to a CNN network for feature extraction; and reshaping data of the feature extraction and transmitting the reshaped data to a LSTM network for classification.

### FIELD

The disclosure relates to a method for identifying driving fatigue, and more particularly, to a method for identifying driving fatigue based on a CNN-LSTM deep learning model.

### BACKGROUND

In today's society, with the development of science and technology and transportation technology, China has made great progress in the field of transportation. However, while enjoying the convenience brought by transportation, traffic accidents are increasing day by day, and a main cause of the accidents is driving fatigue. Therefore, the establishment of a mechanism for effectively monitoring a fatigue state of a driver in real time is an important part of the development of intelligent transportation.

A physiological signal, as the most widely used method to judge the fatigue driving at present, can effectively distinguish the fatigue state of the driver through a physiological difference shown by a body. An electroencephalograph (EEG), an event-related potential (ERP), an electro-oculogram (EOG), an electrocardiograph (ECG) and an electromyography (EMG) are all commonly used measurement indicators based on the physiological signal.

The general study of the ECG (electrocardiograph) mainly focuses on heart rate (HR), and heart rate variability (HRV), both of which are closely related to an autonomic nervous system. The study shows that when the driver is fatigue, the heart rate will slow down and the heart rate variability will change.

The EMG (electromyography) can be recorded by electrodes attached to a muscle surface, which can reflect functional states of nerves and muscles in different states. The study shows that when the driver is fatigue, a frequency and an amplitude of the EMG will change.

When people open and close their eyes, a waveform of the EOG (electro-oculogram) will change obviously, and movements of eyeballs can also provide a fatigue signal. In this way, state of eyes and blink frequency can be analyzed through the change in the waveform of the EOG, so as to reflect an alert level of the brain and detect a fatigue degree of the driver.

The ERP (event-related potential) is a potential induced by external stimulation, which records an electrophysiological response of the brain during information processing on the external stimulation. The ERP is P300, which is the most studied signal, and experiments show that a response speed of the driver to the external stimulation decreases under a fatigue state.

The EEG (electroencephalograph) signal is the most predictive and reliable indicator, which is closely related to a human mental activity, and a physiological activity caused by the driving fatigue is reflected in the EEG Different brain states may have different change rules of the EEG signal, and these features that can represent various states are extracted and classified, such as a power spectral density and an information entropy, so that the fatigue state of the brain can be effectively distinguished.

At the present stage, most classification methods adopt a machine learning method, such as: support vector machine (SVM), artificial neural networks (ANN), decision tree (DT), K-nearest neighbor (KNN), random forest (RF), etc. After preprocessing and feature extraction, the EEG signal is transmitted to an identification model to complete training, so that the trained model can be used to classify data to be tested.

Although many physiological indicators have been proved to be effective in reflecting the fatigue state of the driver, only the EEG signal has a strong accuracy, which is closely related to a mental state of the brain, while other signals similar to the ECG the EMG and the EOG are only external reflection of the body, and there is no way to accurately evaluate the fatigue state of the driver. An external environment has a great influence on eyes of the driver, and there is a certain difficulty to simulate a complexity of a real environment in a simulation experiment. However, a heart rate index in the ECG signal may also be greatly affected by physical exertion. In actual application, there is no stimulation that can induce stable ERP, and if the stimulation is introduced, a main task may be affected to a certain extent. Although the EEG is the best physiological signal reflecting the fatigue state, there are still some defects in analysis and classification methods. The SVM may consume a lot of memory and computation time when dealing with complex data, and similarly, the KNN may also slow down classification speed due to overload of data. Moreover, these classifiers rely strictly on training data instead of general data, and do not make full use of a sequential characteristic of the EEG signal either. In terms of feature extraction, most researches rely on manual extraction, which has a great relationship with a level of the researchers themselves and cannot accurately represent EEG information.

### SUMMARY

In order to solve the technical problems above, the disclosure is intended to provide a method for identifying driving fatigue based on a CNN-LSTM deep learning model, which can be suitable for processing big data, directly acts on original data, automatically performs feature learning layer by layer, and can also express an internal relation and structure of data, so as to improve the detection of driving fatigue of a driver.

The technical solutions adopted in the disclosure are as follows.

There is provided a method for identifying driving fatigue based on a CNN-LSTM deep learning model including the following steps of:

collecting electroencephalograph signals of a subject during simulated driving for a time interval T;

randomly issuing an operating command during simulated driving, and dividing the electroencephalograph signals into fatigue data and non-fatigue data according to a reaction time for the subject to complete the operating command;

performing band-pass filtering and mean removal preprocessing on the electroencephalograph signals, and respectively extracting N minutes of fatigue electroencephalograph signal data and N minutes of non-fatigue electroencephalograph signal data to be detected;

performing independent component analysis on the electroencephalograph signal data to remove interference signals;

establishing a CNN-LSTM model mainly composed of a CNN network and a LSTM network, and setting network parameters of the CNN-LSTM model;

transmitting the electroencephalograph signal data with the interference signals removed to the CNN network for feature extraction; and

reshaping data of the feature extraction and transmitting the reshaped data to the LSTM network for classification.

Further, the dividing the electroencephalograph signals into fatigue data and non-fatigue data includes a rule that, when the reaction time is smaller than θ₁, data before that time point is marked as alert data, when the reaction time is between θ₁ and θ₂, data between two time points respectively corresponding to θ₁ and θ₂ is marked as intermediate state data, and when the reaction time is greater than θ₂, data after that time point is marked as fatigue data.

Further, the thresholds θ₁ and θ₂ are derived from a training experiment, θ₁ is a mean of the reaction times calculated from the beginning of the experiment to the first time for the subject to show a fatigue state or to a time when a driving path of a vehicle deviates from a normal travelling trajectory during the training experiment; θ₂ is a mean of the reaction times during a period when the subject is shown externally to be in a fatigue state or when a driving path of a vehicle deviates from a normal travelling trajectory during the training experiment.

The network parameters of the CNN-LSTM model are respectively as follows: for the CNN network, Convolution_layers is set to be 3 with a parameter of 5*5, and Max-Pooling_layers is set to be 3 with a parameter of 2*2/2; and for the LSTM network, Hidden_Size is set to be 128, Num_Layers is set to be 128, Learning_Rate is set to be 0.001, Batch_Size is set to be 50, and Train_Times is set to be 50. The whole model network consists of 134 layers.

Particularly, before transmitting the electroencephalograph signal data to the CNN network for the feature extraction, a column number is adjusted to meet convolution and pooling requirements.

Further, the feature extraction of the electroencephalograph signal data by the CNN network includes the following steps: a1) performing the feature extraction on the electroencephalograph signal data through the Convolution to obtain a convolution feature output map; a2) pooling the convolution feature map by a max-pooling method to obtain a pooling feature map; and a3) repeating the steps a1) and a2) twice.

Further, max-pooling outputs corresponding to convolution kernels with the same length are connected to form a continuous feature sequence window during the pooling in the step a2); and max-pooling outputs corresponding to different convolution kernels are connected to obtain a plurality of feature sequence windows maintaining an original relative sequence.

Further, the classification by the LSTM network is as follows:

a first layer fₜ is a forget gate, which determines information to be discarded from a cell state;

fₜ=δ(W_{f}[hₜ₋₁,xₜ]+b_{f})

wherein, hₜ₋₁ represents an output from a previous unit, xₜ represents an input to a current unit, fₜ represents an output from the forget gate, δ represents a sigmoid excitation function, and W_{f} and b_{f} represent a weighting term and a bias term respectively;

a second layer iₜ is an input gate and is a sigmoid function, which determines information to be updated;

iₜ=δ(Wᵢ[hₜ₋₁,xₜ]+bᵢ)

wherein, iₜ is used to confirm an update status and add the update status to an update unit, hₜ₋₁ represents an output from a previous unit, xₜ represents an input to a current unit, δ represents a sigmoid excitation function, and Wᵢ and bᵢ represent a weighting term and a bias term respectively;

a third layer Ĉₜ is a tan h layer, which updates a cell state by creating a new candidate vector;

\tilde{C}ₜ=tan h(W_{c}[hₜ₋₁,xₜ]+b_{c})

wherein, Ĉₜ is used to confirm an update status and add the update status to an update unit, hₜ₋₁ represents an output from a previous unit, xₜ represents an input to a current unit, δ represents a sigmoid excitation function, and W_{c} and b_{c} represent a weighting term and a bias term respectively;

the second layer and the third layer work jointly to update a cell state of a neural network module;

a fourth layer Oₜ is a layer for updating other relevant information, which is used to update a change in the cell state caused by other factors;

oₜ=δ(W[hₜ₋₁,xₜ]+bₒ)

wherein, hₜ₋₁ represents an output from a previous unit, χₜ represents an input to a current unit, δ represents a sigmoid excitation function, Wₒ and bₒ represent a weighting term and a bias term respectively, and Oₜ is used as an intermediate term to obtain an output term hₜ with Cₜ; and

Cₜ=fₜ*Cₜ₋₁+iₜ*\tilde{C}ₜ

hₜ=oₜ*tan h(Cₜ)

wherein, fₜ represents an output from the forget gate, iₜ and Ĉₜ are used to confirm an update status and add the update status to an update unit, Cₜ₋₁ is a unit before updating, Cₜ is a unit after updating, and Oₜ is used as an intermediate term to obtain an output term hₜ with Cₜ.

The disclosure has the beneficial effects that: in the disclosure a CNN-LSTM model is constructed by a deep learning method, the CNN network has a strong advantage in processing big and complex data, directly acts on original data when the feature extraction is performed, and automatically performs feature learning layer by layer, so that compared with traditional manual feature extraction, the CNN-LSTM model can better characterize general data without excessively relying on training data. Moreover, the electroencephalograph signals are typical time sequence signals, and the LSTM network can be used for classification to better make use of the time sequence characteristics thereof. Experimental results show that a relatively high accuracy is achieved, which is 96.3±3.1% (total mean value t total standard deviation).

### BRIEF DESCRIPTION OF THE DRAWINGS

The specific embodiments of the disclosure are further described hereinafter with reference to the accompanying drawings.

FIG. 1 is a diagram illustrating electrode placement of an improved 10-20 international system according to the disclosure;

FIG. 2 is a structure diagram of a CNN network according to the disclosure; and

FIG. 3 is a structure diagram of a LSTM network according to the disclosure.

### DETAILED DESCRIPTION OF THE EMBODIMENTS

According to the disclosure, there is provided a method for identifying driving fatigue based on a CNN-LSTM deep learning model including the following steps.

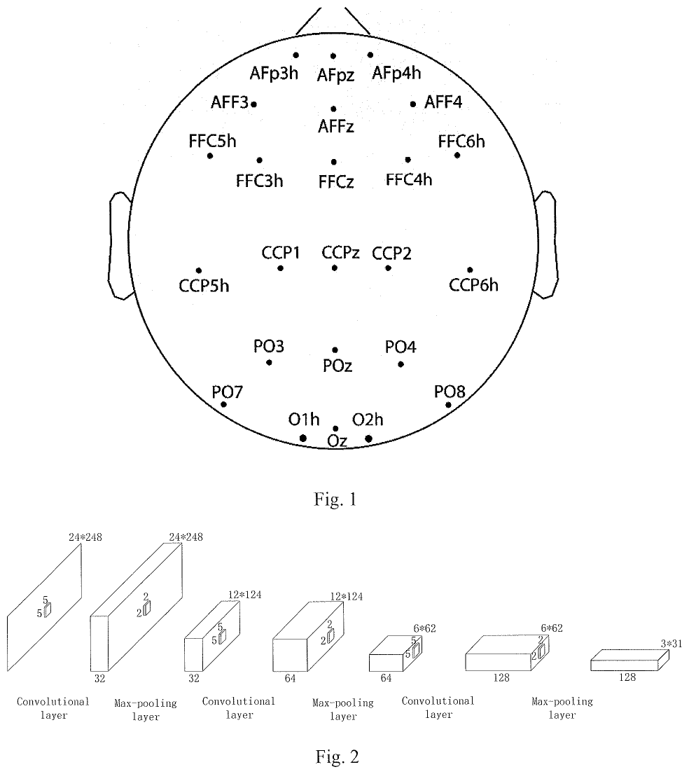

Electroencephalograph signals of a subject during simulated driving are collected for a time interval T. The electroencephalograph signals of the subject during the simulated driving are collected firstly by an electroencephalograph collection apparatus, the time interval adopted in the embodiment is 90 minutes, and electroencephalograph data of 31 subjects are collected in total. Electrodes are placed with an improved 10-20 international standard during electroencephalograph collection, including a total of 24 leads. The electrodes are placed in a manner as shown in FIG. 1.

An operating command is randomly issued during simulated driving, and the electroencephalograph signals are divided into fatigue data and non-fatigue data according to a reaction time for the subject to complete the operating command. Specifically, when the subject performs the simulated driving, a guide vehicle in a screen randomly issues a braking command, a time interval from a time when the subject sees the command to a time when the subject makes a response is recorded, and the reaction time is counted.

When the reaction time is smaller than θ₁, data before that time point is marked as alert data, when the reaction time is between θ₁ and θ₂, data between two time points corresponding to θ₁ and θ₂ respectively is marked as intermediate state data, and when the reaction time is greater than θ₂, data after that time point is marked as fatigue data.

The threshold values are derived from a training experiment, and due to individual difference among the subjects, the time interval threshold values are set differently. Therefore, the time interval threshold values for the individual subjects need to be obtained through the training experiment before a testing experiment. θ₁ is a mean of the reaction times calculated from the beginning of the experiment to the first time for the subject to show a fatigue state (such as yawn) or to the time when a driving path of a vehicle deviates from a normal travelling trajectory during the training experiment. And θ₂ is a mean of the reaction times during a period when the subject is shown externally to be in a fatigue state (such as yawn) or when a driving path of a vehicle deviates from a normal travelling trajectory during the training experiment. In order to ensure that the subjects all enter the fatigue state, changes in the reaction time are counted, and if the reaction time is increased, data is maintained. A sampling frequency for the collected data is 250 Hz.

In order to remove interference signals from the collected data, since the electroencephalograph signals are easily interfered by other signals during extraction, such as electro-oculogram, electrocardiograph, electromyography and power frequency noise, a reasonable algorithm that can remove the interference needs to be designed to improve a signal-to-noise ratio of signals. Therefore, the collected signals are preprocessed in the technical solution. Band-pass filtering at 1 Hz to 30 Hz and mean removal preprocessing are performed on the electroencephalograph signals collected in the stimulated fatigue driving experiment firstly, ten minutes of fatigue electroencephalograph data and ten minutes of non-fatigue electroencephalograph data to be detected are respectively extracted, and then independent component analysis (ICA) is performed on the electroencephalograph data to remove electro-oculogram signal interference (which may also be electrocardiograph, electromyography and power frequency noise). The ICA is processed in electroencephalograph signal data taking a time as a window and having a step size of 5 seconds.

Specifically, the principle for ICA is as follows.

If an unknown original signal s forms a column vector s=(s₁, s₂, . . . , sₘ)^{T}, assuming that at a certain time t, x=(x₁, x₂, . . . , xₙ)^{T} is an n-dimensional random observation column vector, and the following equation is met:

$$x ( t ) = A s ( t ) = {\sum }_{i = 1}^{m} {a}_{i} {s}_{i}$$

wherein, αᵢ represents the i^{th} vector in the m^{th} row of hybrid matrix A, then the ICA is intended to determine a demixing matrix B, so as to obtain that y is optimal approximation of s after x is processed through the demixing matrix B, which can be expressed with a mathematical formula:

y(t)=Bx(t)=BAs(t)

The two sections of fatigue and non-fatigue electroencephalograph signal data for ten minutes respectively preprocessed above are marked as an alert state and a fatigue state respectively by taking a time window as 1 second and a step size as 0.5 second. 70% of the experimental data is used for training and the remaining 30% of the experimental data is used for a classification test.

In order to achieve accurate classification results, it is particularly critical to select a feature that can better characterize data. After feature selection, how to select a classifier is also crucial, because different classifiers have different characteristics, and whether classifier selection is suitable will directly affect the classification results.

Therefore, the CNN-LSTM model is established next, which is composed of two main parts: regional convolution neural network layer (regional CNN) and a long-and-short-term memory neural network layer (LSTM). Although a deep learning network has a strong learning capability, some super-parameters need to be set based on model requirements and manual work experience, so that the algorithm has a faster optimization speed and a higher classification accuracy.

Network Parameters:

(1) Convolution: the Convolution is used for feature extraction, when a convolution kernel has a larger size and more convolution kernels are provided, there will be more features extracted, and meanwhile, the amount of computation will also be increased greatly, with a step size usually set to be 1.

(2) Max-Pooling: the Max-Pooling is used for feature map scaling, which may affect an accuracy of a network.

(3) Hidden_Size: the larger the Hidden_Size is, the stronger the LSTM network is, but calculating parameters and the amount of computation will be increased greatly; and moreover, it shall be noted that the Hidden_Size cannot exceed a number of training samples, otherwise over-fitting is easy to occur.

(4) Learning_Rate: the Learning_Rate may affect update speed of a connection weight for each neuron; the larger the Learning_Rate is, the faster the weight is updated; in the later period of training, a loss function may oscillate around an optimal value, leading to a small Learning_Rate and thus a slow update speed of the weight, and an excessively small weight may lead to a slow descent speed of the optimal loss function.

(5) Num_Layers: when more Num_Layers are provided, the LSTM network becomes larger, and the learning capability becomes stronger, and meanwhile, the amount of computation may also be increased greatly.

(6) Batch_Size: for the Batch_Size, update of a network weight is based on feedback of results of a small-batch training data set, when the Batch_Size is too small, network instability or under-fitting is easily caused, and when the Batch_Size is too large, the amount of computation may be increased greatly.

(7) Train_Times: With the continuously increased Train_Times, the more accurate the network is, but when the Train_Times reach a certain value, accuracy of LSTM network will no longer be improved or will be increased very little, while the amount of computation is continuously increased. Therefore, appropriate Train_Times shall be selected according to requirements for a research problem during specific operation.

Parameter setting of the disclosure is shown in Table 1 below.

TABLE 1
CNN-LSTM Network Parameters

| Network | Parameter | Description | Value |
|---|---|---|---|
| CNN | Convolution_layers | Number of Convolution | 3 |
| | | Networks layer | |
| | Max-Pooling_layers | Number of Max- | 3 |
| | | Pooling Layer | |
| | Convolution | Convolution layer | 5*5 |
| | Max-Pooling | Max-Pooling_layer | 2*2/2 |
| LSTM | Hidden_Size | Number of hidden | 128 |
| | | layer neurons | |
| | Num_Layers | Number of network | 128 |
| | | layers | |
| | Learning_Rate | Learning rate | 0.001 |
| | Batch_Size | Batch size | 50 |
| | Train_Times | Train times | 50 |

After a model of feature extraction and classification is constructed, the preprocessed data may not be able to be feature extracted and classified by the constructed model due to some problems of dimension or other aspects, which requires further processing of the data.

Therefore, the preprocessed data is input into the CNN-LSTM model next, but since the preprocessed electroencephalograph signal data 24*250 cannot be convolved and pooled for 3 times, the last two columns are removed to obtain 24*248, and then the data is input into the CNN network for feature extraction. The CNN network has a structure as shown in FIG. 2. A specific process is as follows.

Firstly, performing feature extraction on the Convolution to obtain a convolution feature output map, then entering the Max-Pooling which ‘discards’ non-maximum values by maximum value operation, reducing the computation amount of the next layer and simultaneously extracting the dependent information in each region. Performing pooling process on the convolution feature map by a max-pooling method to obtain a pooled feature map, connecting maximum pooling outputs corresponding to convolution kernels with the same length to form a continuous sequence to form a window, performing the same operation on the outputs obtained by different convolution kernels to obtain a plurality of windows maintaining the original relative sequence.

After convolution and pooling for three times, a sequence vector in a feature sequence window layer is taken as an input of a next layer of the LSTM network.

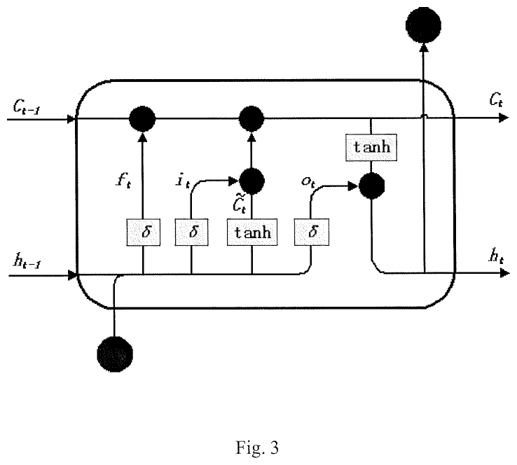

The data after feature extraction output from the CNN network is input to the LSTM network for classification. Since the LSTM network processes time sequence data, 3*31*128 needs to be reshaped into 93*128, that is, a vector with a length of 93 is input for 128 times in total, and a judgment result of the label data is obtained finally. The LSTM network has a structure as shown in FIG. 3.

The LSTM network has a calculation process as follows.

A first layer fₜ is a forget gate, which determines information to be discarded from a cell state;

fₜ=δ(W_{f}[hₜ₋₁,xₜ]+b_{f})

wherein, hₜ₋₁ represents an output from a previous unit, xₜ represents an input to a current unit, fₜ represents an output from the forget gate, δ represents a sigmoid excitation function, and W_{f} and b_{f} represent a weighting term and a bias term respectively.

A second layer iₜ is an input gate and is typically a sigmoid function, which determines information to be updated;

iₜ=δ(Wᵢ[hₜ₋₁,xₜ]+bᵢ)

wherein, iₜ is used to confirm an update status and add the update status to an update unit, hₜ₋₁ represents an output from a previous unit, xₜ represents an input to a current unit, δ represents a sigmoid excitation function, and Wᵢ and bᵢ represent a weighting term and a bias term respectively.

A third layer Ĉₜ is a tan h layer, which updates a cell state by creating a new candidate vector;

\tilde{C}ₜ=tan h(W_{c}[hₜ₋₁,xₜ]+b_{c})

wherein, Ĉₜ is used to confirm an update status and add the update status to an update unit, hₜ₋₁ represents an output from a previous unit, xₜ represents an input to a current unit, δ represents a sigmoid excitation function, and W_{c} and b_{c} represent a weighting term and a bias term respectively.

The second layer and the third layer work jointly to update a cell state of a neural network module.

A fourth layer Oₜ is a layer for updating other relevant information, which is used to update a change in the cell state caused by other factors;

oₜ=δ(Wₒ[hₜ₋₁,xₜ]+bₒ)

wherein, hₜ₋₁ represents an output from a previous unit, xₜ represents an input to a current unit, δ represents a sigmoid excitation function, Wₒ and bₒ represent a weighting term and a bias term respectively, and Oₜ is used as an intermediate term to obtain an output term hₜ with Cₜ.

Cₜ=fₜ*Cₜ₋₁+iₜ*\tilde{C}ₜ

hₜ=oₜ*tan h(Cₜ)

wherein, fₜ represents an output from the forget gate, iₜ and Ĉₜ are used to confirm an update status and add the update status to an update unit, Cₜ₋₁ is a unit before updating, Cₜ is a unit after updating, and Oₜ is used as an intermediate term to obtain an output term hₜ with Cₜ.

Using the model, 5 experiments were performed and the mean and standard deviation were calculated to achieve a classification accuracy of 96.3+−3.1% (total mean±total standard deviation), as detailed in Table 2.

TABLE 2
Classification Accuracy of Each Subject and Total Classification Accuracy

| | | | | | | | | | Total |
|---|---|---|---|---|---|---|---|---|---|
| | | | | | | Mean | Standard | Grand | standard |
| Subjects | Experiment 1 | Experiment 2 | Experiment 3 | Experiment 4 | Experiment 5 | value | deviation | mean | deviation |
| Subject 1 | 0.9806 | 0.9875 | 0.9875 | 0.9819 | 0.9903 | 0.986 | 0.004 | 0.963 | 0.031 |
| Subject 2 | 0.9222 | 0.9181 | 0.9028 | 0.9097 | 0.9111 | 0.913 | 0.008 | | |
| Subject 3 | 0.9722 | 0.9653 | 0.9778 | 0.975 | 0.9778 | 0.974 | 0.005 | | |
| Subject 4 | 0.9528 | 0.9417 | 0.9375 | 0.9333 | 0.9569 | 0.944 | 0.010 | | |
| Subject 5 | 0.9889 | 0.9819 | 0.9861 | 0.9806 | 0.9736 | 0.982 | 0.006 | | |
| Subject 6 | 0.9444 | 0.9458 | 0.9514 | 0.9292 | 0.9389 | 0.942 | 0.008 | | |
| Subject 7 | 0.8847 | 0.9097 | 0.8667 | 0.9028 | 0.8833 | 0.889 | 0.017 | | |
| Subject 8 | 0.9833 | 0.9931 | 0.9861 | 0.9958 | 0.9847 | 0.989 | 0.006 | | |
| Subject 9 | 0.9889 | 0.9944 | 0.9875 | 0.9944 | 0.9861 | 0.990 | 0.004 | | |
| Subject 10 | 0.9722 | 0.9764 | 0.9514 | 0.9625 | 0.9681 | 0.966 | 0.010 | | |
| Subject 11 | 0.9597 | 0.9319 | 0.9236 | 0.9181 | 0.9375 | 0.934 | 0.016 | | |
| Subject 12 | 0.9472 | 0.9528 | 0.975 | 0.9653 | 0.9667 | 0.961 | 0.011 | | |
| Subject 13 | 0.9569 | 0.9708 | 0.975 | 0.9639 | 0.975 | 0.968 | 0.008 | | |
| Subject 14 | 0.9875 | 0.9875 | 0.9889 | 0.9833 | 0.9944 | 0.988 | 0.004 | | |
| Subject 15 | 0.9583 | 0.9667 | 0.9583 | 0.9722 | 0.9694 | 0.965 | 0.006 | | |
| Subject 16 | 0.9944 | 0.9958 | 0.9958 | 0.9958 | 0.9917 | 0.995 | 0.002 | | |
| Subject 17 | 0.9931 | 0.9917 | 0.9861 | 0.9903 | 0.9931 | 0.991 | 0.003 | | |
| Subject 18 | 0.9944 | 0.9931 | 0.9944 | 0.9931 | 0.9972 | 0.994 | 0.002 | | |
| Subject 19 | 0.9889 | 0.9792 | 0.9806 | 0.9903 | 0.9861 | 0.985 | 0.005 | | |
| Subject 20 | 0.9889 | 0.9889 | 0.9819 | 0.9806 | 0.9903 | 0.986 | 0.005 | | |
| Subject 21 | 0.9569 | 0.9264 | 0.9222 | 0.9486 | 0.9264 | 0.936 | 0.016 | | |
| Subject 22 | 0.9583 | 0.9556 | 0.9694 | 0.9667 | 0.95 | 0.960 | 0.008 | | |
| Subject 23 | 0.8458 | 0.8736 | 0.8639 | 0.9139 | 0.8778 | 0.875 | 0.025 | | |
| Subject 24 | 0.9486 | 0.9333 | 0.9528 | 0.9639 | 0.9639 | 0.953 | 0.013 | | |
| Subject 25 | 0.9694 | 0.95 | 0.9667 | 0.9514 | 0.9625 | 0.960 | 0.009 | | |
| Subject 26 | 0.9625 | 0.9569 | 0.9681 | 0.9681 | 0.9417 | 0.959 | 0.011 | | |
| Subject 27 | 0.9847 | 0.9903 | 0.9847 | 0.9875 | 0.9889 | 0.987 | 0.003 | | |
| Subject 28 | 0.9653 | 0.95 | 0.975 | 0.9514 | 0.9583 | 0.960 | 0.010 | | |
| Subject 29 | 0.9403 | 0.9792 | 0.9833 | 0.9681 | 0.9736 | 0.969 | 0.017 | | |
| Subject 30 | 0.9806 | 0.9708 | 0.9569 | 0.975 | 0.9708 | 0.971 | 0.009 | | |
| Subject 31 | 0.9944 | 0.9972 | 0.9931 | 0.9889 | 0.9917 | 0.993 | 0.003 | | |

The above description is only preferred embodiments of the disclosure, and the disclosure is not limited to the above embodiments. The technical solutions achieving the objectives of the disclosure by basically the same means shall all fall within the protection scope of the disclosure.

What is claimed is:

1. A method for identifying driving fatigue based on a CNN-LSTM deep learning model, comprising the following steps of:
collecting electroencephalograph signals of a subject during simulated driving for a time interval T;
randomly issuing an operating command during simulated driving, and dividing the electroencephalograph signals into fatigue data and non-fatigue data according to a reaction time for the subject to complete the operating command;
performing band-pass filtering and mean removal preprocessing on the electroencephalograph signals, and respectively extracting N minutes of fatigue electroencephalograph signal data and N minutes of non-fatigue electroencephalograph signal data to be detected;
performing independent component analysis on the electroencephalograph signal data to remove interference signals;
establishing a CNN-LSTM model mainly composed of a CNN network and a LSTM network, and setting network parameters of the CNN-LSTM model;
transmitting the electroencephalograph signal data with interference signals removed to the CNN network for feature extraction; and
reshaping data of the feature extraction and transmitting the reshaped data to the LSTM network for classification.

2. The method for identifying driving fatigue based on a CNN-LSTM deep learning model according to claim 1, wherein the dividing the electroencephalograph signals into fatigue data and non-fatigue data comprises a rule that, when the reaction time is smaller than θ₁, data before that time point is marked as alert data, when the reaction time is between θ₁ and θ₂, data between two time points respectively corresponding to θ₁ and θ₂ is marked as intermediate state data, and when the reaction time is greater than θ₂, data after that time point is marked as fatigue data.

3. The method for identifying driving fatigue based on a CNN-LSTM deep learning model according to claim 2, wherein thresholds θ₁ and θ₂ are derived from a training experiment, θ₁ is a mean of the reaction times calculated from the beginning of the experiment to the first time for the subject to show a fatigue state or to a time when a driving path of a vehicle deviates from a normal travelling trajectory during the training experiment; θ₂ is a mean of the reaction times during a period when the subject is shown externally to be in a fatigue state or when a driving path of a vehicle deviates from a normal travelling trajectory during the training experiment.

4. The method for identifying driving fatigue based on a CNN-LSTM deep learning model according to claim 1, wherein the network parameters of the CNN-LSTM model are respectively as follows: for the CNN network, Convolution_layers is set to be 3 with a parameter of 5*5, and Max-Pooling_layers is set to be 3 with a parameter of 2*2/2; and for the LSTM network, Hidden_Size is set to be 128, Num_Layers is set to be 128, Learning_Rate is set to be 0.001, Batch_Size is set to be 50, and Train_Times is set to be 50.

5. The method for identifying driving fatigue based on a CNN-LSTM deep learning model according to claim 1, wherein before transmitting the electroencephalograph signal data to the CNN network for feature extraction, a column number is adjusted to meet convolution and pooling requirements.

6. The method for identifying driving fatigue based on a CNN-LSTM deep learning model according to claim 1, wherein the feature extraction of the electroencephalograph signal data by the CNN network comprises the following steps: a1) performing the feature extraction on the electroencephalograph signal data through the Convolution to obtain a convolution feature output map; a2) pooling the convolution feature map by a max-pooling method to obtain a pooling feature map; and a3) repeating the steps a1) and a2) twice.

7. The method for identifying driving fatigue based on a CNN-LSTM deep learning model according to claim 6, wherein max-pooling outputs corresponding to convolution kernels with the same length are connected to form a continuous feature sequence window during the pooling in the step a2); and max-pooling outputs corresponding to different convolution kernels are connected to obtain a plurality of feature sequence windows maintaining an original relative sequence.

8. The method for identifying driving fatigue based on a CNN-LSTM deep learning model according to claim 4, wherein the feature extraction of the electroencephalograph signal data by the CNN network comprises the following steps: a1) performing the feature extraction on the electroencephalograph signal data through the Convolution to obtain a convolution feature output map; a2) pooling the convolution feature map by a max-pooling method to obtain a pooling feature map; and a3) repeating the steps a1) and a2) twice.

9. The method for identifying driving fatigue based on a CNN-LSTM deep learning model according to claim 8, wherein max-pooling outputs corresponding to convolution kernels with the same length are connected to form a continuous feature sequence window during the pooling in the step a2); and max-pooling outputs corresponding to different convolution kernels are connected to obtain a plurality of feature sequence windows maintaining an original relative sequence.

10. The method for identifying driving fatigue based on a CNN-LSTM deep learning model according to claim 5, wherein the feature extraction of the electroencephalograph signal data by the CNN network comprises the following steps: a1) performing the feature extraction on the electroencephalograph signal data through the Convolution to obtain a convolution feature output map; a2) pooling the convolution feature map by a max-pooling method to obtain a pooling feature map; and a3) repeating the steps a1) and a2) twice.

11. The method for identifying driving fatigue based on a CNN-LSTM deep learning model according to claim 10, wherein max-pooling outputs corresponding to convolution kernels with the same length are connected to form a continuous feature sequence window during the pooling in the step a2); and max-pooling outputs corresponding to different convolution kernels are connected to obtain a plurality of feature sequence windows maintaining an original relative sequence.

12. The method for identifying driving fatigue based on a CNN-LSTM deep learning model according to claim 1, wherein the classification by the LSTM network is as follows:
a first layer fₜ is a forget gate, which determines information to be discarded from a cell state;
fₜ=δ(W_{f}[hₜ₋₁,xₜ]+b_{f})
wherein, hₜ₋₁ represents an output from a previous unit, xₜ represents an input to a current unit, fₜ represents an output from the forget gate, δ represents a sigmoid excitation function, and W_{f} and b_{f} represent a weighting term and a bias term respectively;
a second layer iₜ is an input gate and is a sigmoid function, which determines information to be updated;
iₜ=δ(Wᵢ[hₜ₋₁,xₜ]+bᵢ)
wherein, iₜ is used to confirm an update status and add the update status to an update unit, hₜ₋₁ represents an output from a previous unit, xₜ represents an input to a current unit, δ represents a sigmoid excitation function, and Wᵢ and bᵢ represent a weighting term and a bias term respectively;
a third layer Ĉₜ is a tan h layer, which updates a cell state by creating a new candidate vector;
\tilde{C}ₜ=tan h(W_{c}[hₜ₋₁,xₜ]+b_{c})
wherein, Ĉₜ is used to confirm an update status and add the update status to an update unit, hₜ₋₁ represents an output from a previous unit, xₜ represents an input to a current unit, δ represents a sigmoid excitation function, and W_{c} and b_{c} represent a weighting term and a bias term respectively;
the second layer and the third layer work jointly to update a cell state of a neural network module;
a fourth layer Oₜ is a layer for updating other relevant information, which is used to update a change in the cell state caused by other factors;
oₜ=δ(Wₒ[hₜ₋₁,xₜ]+bₒ)
wherein, hₜ₋₁ represents an output from a previous unit, y represents an input to a current unit, δ represents a sigmoid excitation function, Wₒ and bₒ represent a weighting term and a bias term respectively, and Oₜ is used as an intermediate term to obtain an output term hₜ with Cₜ; and
Cₜ=fₜ*Cₜ₋₁+iₜ*\tilde{C}ₜ
hₜ=oₜ*tan h(Cₜ)
wherein, fₜ represents an output from the forget gate, iₜ and Ĉₜ are used to confirm an update status and add the update status to an update unit, Cₜ₋₁ is a unit before updating, Cₜ is a unit after updating, and Oₜ is used as an intermediate term to obtain an output term hₜ with Cₜ.
